SEQUENCE LISTING (24 sequences)

SEQ ID NO: 1 - protein, 111 aa, Vibrio parahaemolyticus
MSNNIKHETDYSHDWTVEPNGGVTEVDSKHTPIIPEVGRSVDIENTGRGELTIQYQWGAPFMAGGWKVAKSHVVQRDETYHLQRPDNAFYHQRIVVINNGASRGFCTIYYH

SEQ ID NO: 2 - protein, 438 aa, Vibrio parahaemolyticus
MTNEYVVTMSSLTEFNPNNARKSYLFDNYEVDPNYAFKAMVSFGLSNIPYAGGFLSTLWNIFWPNTPNEPDIENIWEQLRDRIQDLVDESIIDAINGILDSKIKETRDKIQDINETIENFGYAAAKDDYIGLVTHYLIGLEENFKRELDGDEWLGYAILPLLATTVSLQITYMACGLDYKDEFGFTDSDVHKLTRNIDKLYDDVSSYITELAAWADNDSYNNANQDNVYDEVMGARSWCTVHGFEHMLIWQKIKELKKVDVFVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 3 - protein, 176 aa, Vibrio parahaemolyticus
VHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 4 - protein, 199 aa, Vibrio parahaemolyticus
MKKWIYVVLVLSIAGIGGFSVHAVHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 5 - protein, 176 aa, Vibrio parahaemolyticus
VHSNLISYSPAVGSPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKPSVRLQLEGHFICGMLADQEGSDKVAAFSVAYELFHPDEFGTEK

SEQ ID NO: 6 - protein, 176 aa, Vibrio parahaemolyticus
VHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVITNGPEAIDRIVFHFSDDRTFVVGENSGKPSVRLQLEGHFICGMLADQEGSDKVAAFSVAYELFHPDEFGTEK

SEQ ID NO: 7 - protein, 176 aa, Vibrio parahaemolyticus
VHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKYSVRLQLEGHFICGMLADQEGSDKVAAFSVAYELFHPDEFGTEK

SEQ ID NO: 8 - protein, 176 aa, Vibrio parahaemolyticus
VHSNLISYSPAVGSPSGNFNYIATGTEDEIPQPLKPNMFGERRNRIVKIESWNSIEIHYYNRVGRLKLTYENGEVVELGKAHKYDEHYQSIELNGAYIKYVDVITNGPEAIDRIVFHFSDDRTFVVGENSGKPSVRLQLEGHFICGMLADQEGSDKVAAFSVAYELFHPDEFGTEK

SEQ ID NO: 9 - protein, 176 aa, Vibrio parahaemolyticus
VHSNLISYSPAVGSPSGNFNYIATGTEDEIPQPLKPNMFGERRNRIVKIESWNSIEIHYYNRVGRLKLTYENGEVVELGKAHKYDEHYQSIELNGAYIKYVDVIANGPEAIDRIVFHFSDDRTFVVGENSGKYSVRLQLEGHFICGMLADQEGSDKVAAFSVAYELFHPDEFGTEK

SEQ ID NO: 10 - protein, 176 aa, Vibrio parahaemolyticus
VHSNLISYSPAVGFPSGNFNYIATGTEDEIPQPLKPNMFGERRNRIVKIESWNSIEIHYYNRVGRLKLTYENGEVVELGKAHKYDEHYQSIELNGAYIKYVDVITNGPEAIDRIVFHFSDDRTFVVGENSGKYSVRLQLEGHFICGMLADQEGSDKVAAFSVAYELFHPDEFGTEK

SEQ ID NO: 11 - protein, 176 aa, Vibrio parahaemolyticus
VHSNLISYSPAVGSPSGNFNYIATGTEDEIPQPLKPNMFGERRNRIVKIESWNSIEIHYYNRVGRLKLTYENGEVVELGKAHKYDEHYQSIELNGAYIKYVDVITNGPEAIDRIVFHFSDDRTFVVGENSGKYSVRLQLEGHFICGMLADQEGSDKVAAFSVAYELFHPDEFGTEK

SEQ ID NO: 12 - DNA, 531 bp, Vibrio parahaemolyticus
gttcacagtaatttaatttcatattcacctgctgttggttttcctagtggtaatttcaactatattgctacaggtacggaagatgaaatacctcaaccattaaaaccaaatatgtttggggaacgtcgaaatcgtattgtaaaaattgaatcatggaacagtattgaaatacattattacaatcgcgtaggtcgacttaaactaacttatgaaaatggggaagtggtagaactaggcaaggctcataaatatgacgagcattaccaatctattgagttaaacggcgcttacattaaatatgttgatgttattgccaatggacctgaagcaattgatcgaatcgtatttcatttttcagatgatcgaacatttgttgttggtgaaaactcaggcaagccaagtgtgcgtttgcaactggaaggtcattttatttgtggcatgcttgcggatcaagaaggttctgacaaagttgccgcgtttagcgtggcttatgaattgtttcatcccgatgaatttggtacagaaaagtag

SEQ ID NO: 13 - DNA, 600 bp, Vibrio parahaemolyticus
atgaaaaaatggatatatgttgtgcttgtgctgagtattgcagggatcggcggcttctccgtccacgcagttcacagtaatttaatttcatattcacctgctgttggttttcctagtggtaatttcaactatattgctacaggtacggaagatgaaatacctcaaccattaaaaccaaatatgtttggggaacgtcgaaatcgtattgtaaaaattgaatcatggaacagtattgaaatacattattacaatcgcgtaggtcgacttaaactaacttatgaaaatggggaagtggtagaactaggcaaggctcataaatatgacgagcattaccaatctattgagttaaacggcgcttacattaaatatgttgatgttattgccaatggacctgaagcaattgatcgaatcgtatttcatttttcagatgatcgaacatttgttgttggtgaaaactcaggcaagccaagtgtgcgtttgcaactggaaggtcattttatttgtggcatgcttgcggatcaagaaggttctgacaaagttgccgcgtttagcgtggcttatgaattgtttcatcccgatgaatttggtacagaaaagtag

SEQ ID NO: 14 - protein, 23 aa, Bacillus subtilis
MKKWIYVVLVLSIAGIGGFSVHA

SEQ ID NO: 15 - DNA, 69 bp, Bacillus subtilis
atgaaaaaatggatatatgttgtgcttgtgctgagtattgcagggatcggcggcttctccgtccacgca

SEQ ID NO: 16 - protein, 23 aa, Vibrio parahaemolyticus
WADNDSYNNANQDNVYDEVMGAR

SEQ ID NO: 17 - protein, 13 aa, Vibrio parahaemolyticus
WADNDSYNNANQD

SEQ ID NO: 18 - protein, 16 aa, Vibrio parahaemolyticus
FVVGENSGKPSVRLQL

SEQ ID NO: 19 - protein, 10 aa, Vibrio parahaemolyticus
SGKPSVRLQL

SEQ ID NO: 20 - protein, 15 aa, Vibrio parahaemolyticus
CVQRDETYHLQRPDN

SEQ ID NO: 21 - DNA, 21 bp, Artificial Sequence
  Ap4-F1 primer
gtggtaatagattgtacagaa

SEQ ID NO: 22 - DNA, 21 bp, Artificial Sequence
  Ap3R Primer
gtggtaatagattgtacagaa

SEQ ID NO: 23 - DNA, 20 bp, Artificial Sequence
  Vp-gyrB for primer
cgagcatgcgctaagtgttg

SEQ ID NO: 24 - DNA, 20 bp, Artificial Sequence
  Vp-gyrB-rev
taacgctgacggcttagacc

(19) United States
(12) Patent Application Publication
(10) Pub. No.: US 2022/0313783 A1
(43) Pub. Date: Oct. 6, 2022

(54) Systems, Methods and Compositions for the Inhibition of Bacterial Toxins to Treat Early Mortality Syndrome in Aquatic Animals

(71) Applicant: Pebble Labs Inc., Los Alamos, NM (US)

(72) Inventors: Tatiana Vinogradova-Shah, Los Alamos, NM (US); Elena Sineva, Los Alamos, NM (US); Tim Travers, White Rock, NM (US); Eugene Parsons, Port St. Lucie, FL (US); Diana Ballesteros, White Rock, NM (US); Richard Sayre, Los Alamos, NM (US); Sean Flattery, Freeland, MI (US)

(21) Appl. No.: 17/639,486

(22) PCT Filed: Sep. 4, 2020
(86) PCT No.: PCT/US20/49478

§ 371 Date: Mar. 1, 2022

Related U.S. Application Data
(60) Provisional application No. 62/896,034, filed on Sep. 5, 2019.

(51) Int. Cl. A61K 38/17; A61K 47/64; A61K 35/74; A61P 43/00

(52) U.S. Cl. CPC A61K 38/1774; A61K 47/6425; A61K 35/74; A61P 43/00

(57) ABSTRACT

The invention relates to novel systems, methods, and compositions for the competitive inhibition of bacterial toxins expressed in animal systems, and preferably the inhibition of toxins produced by pathogenic bacteria that affect aquatic animals. One aspect of the invention includes methods and compositions for the treatment of Early-Mortality Syndrome (EMS) in shrimp through the use of truncated PirB^{Vp} peptides used as competitor inhibitors to reduce formation of the cytotoxic PirA^{Vp}/PirB^{Vp} dimer complex.

This application claims the benefit of and priority to U.S. Provisional Application No. 62/896,034, filed Sep. 5, 2020. The entire specification and figures of the above-referenced application are hereby incorporated, in their entirety by reference.

### SEQUENCE LISTING

The instant application contains a Sequence Listing which has been submitted electronically in ASCII format and is hereby incorporated by reference in its entirety. Said ASCII copy, created on Sep. 4, 2020, is named “90115-00491-Sequence-Listing-AF.txt” and is 22.9 Kbytes in size.

### TECHNICAL FIELD

Generally, the inventive technology relates to novel systems, methods, and compositions for the competitive inhibition of bacterial toxins expressed in animal systems, and preferably the inhibition of toxins produced by pathogenic bacteria that affect aquatic animals.

### BACKGROUND

Acute Hepatopancreatic Necrosis Disease (AHND), also known as Early Mortality Syndrome (EMS) has emerged as one of the most devastating diseases affecting shrimp aquaculture. EMS has severely affected the aquaculture industries in several countries in the eastern and western hemispheres such as China, Vietnam, Malaysia, Thailand, and Mexico. In some instances, outbreaks of EMS have result in a staggering 80% loss of shrimp aquaculture populations. EMS is caused by Vibrio bacterial species, which can be transmitted orally. These Vibrio species colonize the shrimp gastrointestinal tract and produce a toxin that causes tissue destruction and dysfunction of the shrimp digestive organ known as the hepatopancreas. EMS typically affects post-larvae shrimp within 20-30 days after stocking and frequently causes up to 100% mortality. Currently, there are no available methods to treat EMS. Traditional strategies to prevent or treat outbreaks of EMS may actually have the effect of aggravating disease propagation. For example, attempts at total disinfection of pond bottom and water to kill possible vectors of EMS may actually contribute to the epidemic spread of the EMS disease rather than control it by removing potentially competitive microbial populations.

As noted above, Vibrio parahaemolyticus (or Vibrio sp.) is a causative strain of EMS and harbors a plasmid that encodes a two gene-operon named PirAB^{Vp}. The PirA^{Vp} and PirB^{Vp} toxins, generally referred to herein as PirA and PirB respectively, are structurally similar to the insecticidal PirA and PirB toxins from Photorhabidus. Both toxins are produced in the shrimp stomach and cause shrimp death by disruption of hepatopancreatic epithelial cells. PirA or PirB separately do not cause mortality and exhibit animal toxicity only in combination as the PirAB binary toxin. Thus, recombinant PirB toxin alone does not cause shrimp disease or mortality even though PirB is proposed to contain both membrane pore-forming and receptor binding domains. As shown in FIG. 1A, to become an active toxin, PirA and PirB must interact with each other. While the complex formation of PirA/PirB has been confirmed, it is not yet fully known how these two toxins bind to each other. Preventing PirA/PirB interactions with the help of probiotic bacteria that continually deliver one or more inhibitor molecules could be a valuable and economically sustainable EMS-disease prevention strategy.

### SUMMARY OF THE INVENTION

Generally, the inventive technology relates to novel strategies for controlling disease-causing agents. One aim of the current invention may include the inactivation of toxins generated by target pathogens, resulting in the suppression of bacterial toxins and/or pathogenic activity of the bacteria in a host organism. In one aspect, the inventive technology may include novel systems, methods, and compositions for treating or preventing Early Mortality Syndrome (EMS) through the use of genetically engineered donor microorganisms, such as bacteria, expressing modified toxin peptides or similar recombinant peptides able to bind to functional sites of toxin protein that may competitively inhibit and/or deactivate toxins produced of pathogenic Vibrio sp. that cause EMS.

The present invention also relates to the utilization of genetically modified donor bacteria that may be configured to produce modified toxin peptides that may competitively inhibit toxin activity in eukaryotic systems. These modified toxin peptides may be further altered to competitively inhibit the activity of their corresponding wild-type toxins produced by a disease-causing agent. In one preferred embodiment, the invention may include compositions and methods for inhibiting the activity of toxins produced by pathogenic bacteria, for example EMS-causing Vibrio species. In one preferred aspect, the invention involves the generation of genetically engineered bacteria, and preferably bacteria that are shrimp symbiotic, endosymbiotic or probiotics, configured to competitively inactivate toxins produced by Vibrio sp., which are known to be causative agents of EMS.

In one aspect, the invention may include a modified PirB toxin configured to inhibit the activity of the formation of the PirA/PirB dimer complex (sometimes referred to as a Pir, or Pir binary toxin (PirA/PirB)) produced by EMS causing Vibrio sp. In one preferred aspect, a modified PirB toxin may include a truncated PirB peptide, and preferably a truncated PirB encoding the protein-protein interface residues, namely amino acid residues 263-438 between PirA and PirB. In this preferred aspect, a modified PirB toxin may include a truncated PirB Δ1-262 peptide, which may further be coupled with a secretion signal domain, such as an YbxI secretion signal.

In another preferred aspect, a modified PirB toxin may include a truncated PirB peptide, and preferably a truncated PirB Δ1-262 peptide that may include one more point mutations at positions 276, 367 or 395 that increase the binding affinity of the truncated PirB Δ1-262 peptide with PirA. In this preferred aspect, the truncated PirB Δ1-262 peptide may include one more of the following point mutations selected from the group consisting of: F276S, A367T, P395Y, or any combination thereof. In another preferred embodiment, the truncated PirB Δ1-262 peptide may include one more of the following combinations of point mutations selected from the group consisting of: F276S/A367T, F276S/P395Y, A367T/P395Y, and F276S/A367T/P395Y.

In another preferred aspect, a modified PirB toxin may include a PirB peptide fragment configured to competitively inhibit the formation of the PirA/PirB dimer complex. In this preferred aspect, a PirB peptide fragment may include all or a portion of a binding interface with PirA.

In another aspect the invention includes systems, methods, and compositions for treating or preventing EMS in aquatic animals, such as shrimp, through the use of genetically engineered bacteria expressing one or more modified PirB peptides configured to competitively inhibit the activity of wild-type the formation of the PirA/PirB dimer complex, which as noted above forms a pathogenic binary bacterial toxin. In this preferred aspect, the invention includes methods of treating EMS in an aquatic animal comprising administering a therapeutically effective amount of a truncated PirB peptide and/or a PirB peptide fragment to an aquatic animal, and preferably a shrimp that is infected by, or susceptible to infection by said EMS-causing bacterial pathogen. In this aspect, a therapeutically effective amount of modified PirB peptide, such as a truncated PirB peptide and/or a PirB peptide fragment, may be administered directly to the target animal, or may be administered through a donor bacteria engineered to express a PirB peptide fragment.

Another aspect the invention includes the generation of treated feed or liquid inoculum containing genetically modified bacteria, or spores of the same, configured to express a modified PirB peptide configured to competitively inhibit the activity of wild-type the formation of the PirA/PirB dimer complex produced by Vibrio populations and thereby prevent or treat the effects of EMS. The treated feed or liquid inoculum may be introduced to a pathogen-susceptible or pathogen-affected population, preferably an aquatic animal such as shrimp grown in aquaculture.

Another aspect the invention includes the expression of modified PirB peptides by genetically modified bacteria may act as a prophylactic protection or vaccine to immunize shrimp against pathogen produced toxins. As such, one aspect of the invention may include the use of genetically modified bacteria to colonize and continuously express modified PirB peptides configured to competitively inhibit the activity of wild-type the formation of the PirA/PirB dimer complex, thereby providing individual or herd immunity in aquatic animals directed to EMS, such as shrimp populations grown in aquaculture systems.

Additional aspects of the invention may include one or more of the following preferred embodiments:

1. A composition for the treatment of Early Mortality Syndrome (EMS) in an aquatic organism comprising a modified PirB peptide, wherein said modified PirB peptide competitively inhibits the formation of the PirA/PirB dimer complex.
2. The composition of claim 1, wherein said modified PirB peptide comprises a truncated PirB peptide.
3. The composition of claim 2, wherein said truncated PirB peptide comprises a PirB Δ1-262 peptide.
4. The composition of any of embodiment 2-3, wherein said truncated PirB peptide comprises the amino acid sequence according to SEQ ID NO. 3.
5. The composition of any of embodiment 2-4, wherein said truncated PirB peptide is coupled with a secretion signal domain.
6. The composition of any of embodiment 2-5, wherein said truncated PirB peptide coupled with a secretion signal domain comprises a truncated PirB peptide coupled with an YbxI secretion signal.
7. The composition of any of embodiment 2-5, wherein said truncated PirB peptide coupled with a secretion signal domain comprises the amino acid sequence according to SEQ ID NO. 4.
8. The composition of any of embodiment 5-7, wherein said secretion signal domain comprises a secretion signal according to SEQ ID NO. 14.
9. The composition of any of embodiment 2-8, wherein the truncated PirB peptide further comprises a truncated PirB peptide having one or more mutations selected from the group consisting of: F276S, A367T, P395Y, or any combination thereof.
10. The composition of any of embodiment 2-8, wherein the truncated PirB peptide further comprises a truncated PirB peptide selected from the group consisting of: SEQ ID NOs. 5-11.
11. The composition of claim 1, wherein said PirA/PirB complex comprises a dimer complex wherein PirA comprises a sequence according to SEQ ID NO. 1, and PirB comprises a sequence according to SEQ ID NO. 2.
12. The composition of claim 1, wherein EMS is caused by a Vibrio sp.
13. The composition of claim 1, wherein said aquatic organism comprises shrimp.
14. A method of treating EMS in an aquatic animal comprising administering a therapeutically effective amount of a modified PirB peptide of any of embodiment 1-11 to an aquatic animal that is infected by, or susceptible to infection by an EMS-causing bacterial pathogen, wherein said modified PirB peptide competitively inhibits the formation of the PirA/PirB dimer complex.
15. The method of claim 14, wherein said aquatic organism comprises shrimp.
16. The method of claim 14, wherein administering comprises administering a therapeutically effective amount of a donor bacteria engineered to express a truncated PirB peptide of any of embodiment 1-11.
17. The method of claim 14, wherein administering comprises administering a therapeutically effective amount of a donor bacteria engineered to express a truncated PirB peptide of any of embodiment 1-11, wherein said donor bacteria is incorporated into a treated feed or liquid inoculum.
18. The method of claim 17, wherein the donor bacteria comprises a probiotic donor bacteria.
19. The method of claim 18, wherein said probiotic donor bacteria comprises Bacillus subtilis.
20. A method of treating Early Mortality Syndrome (EMS) in an aquatic organism comprising the steps of:
generating a donor microorganism to express a heterologous polynucleotide operably linked to a promoter encoding a modified PirB peptide configured to competitively inhibit the formation of the PirA/PirB dimer complex produced by an EMS-causing bacterial pathogen;
introducing said genetically modified donor microorganism to a target host that is infected by, or susceptible to infection by said EMS-causing bacterial pathogen;
expressing said heterologous modified PirB peptide; and
inhibiting the formation of the PirA/PirB dimer complex produced by an EMS-causing bacterial pathogen.
   21. The method of claim 20, wherein said modified PirB peptide comprises a truncated PirB peptide.
   22. The method of claim 21, wherein said truncated PirB peptide comprises a PirB Δ1-262 peptide.
   23. The method of any of embodiment 21-22, wherein said truncated PirB peptide comprises the amino acid sequence according to SEQ ID NO. 3.
   24. The method of any of embodiment 22-23, wherein said truncated PirB peptide is coupled with a secretion signal domain.
   25. The method of any of embodiment 22-24, wherein said truncated PirB peptide coupled with a secretion signal domain comprises a truncated PirB peptide coupled with an YbxI secretion signal.
   26. The method of any of embodiment 22-24, wherein said truncated PirB peptide coupled with a secretion signal domain comprises the amino acid sequence according to SEQ ID NO. 4.
   27. The method of any of embodiment 24-27, wherein said secretion signal domain comprises a secretion signal according to SEQ ID NO. 14.
   28. The method of any of embodiment 22-27, wherein the truncated PirB peptide further comprises a truncated PirB peptide having one or more mutations selected from the group consisting of: F276S, A367T, P395Y, or any combination thereof.
   29. The method of any of embodiment 22-27, wherein the truncated PirB peptide further comprises a truncated PirB peptide selected from the group consisting of: SEQ ID NOs. 5-11.
   30. The method of claim 20, wherein said PirA/PirB complex comprises a dimer complex wherein PirA comprises a sequence according to SEQ ID NO. 1, and PirB comprises a sequence according to SEQ ID NO. 2.
   31. The method of claim 20, wherein EMS-causing bacterial pathogen comprises a Vibrio sp.
   32. The method of claim 20, wherein said aquatic organism comprises shrimp.
   33. The method of claim 20, wherein said donor microorganism comprises a donor bacteria.
   34. The method of claim 33, wherein said donor bacteria comprises Bacillus subtilis.
   35. A composition for the treatment of Early Mortality Syndrome (EMS) in an aquatic organism comprising a PirB peptide fragment, wherein said PirB peptide fragment competitively inhibits the formation of the PirA/PirB dimer complex.
   36. The composition of claim 35, wherein said PirB peptide fragment comprises a PirB peptide fragment encoding a portion of a binding interface with PirA.
   37. The composition of any of embodiment 35-36, wherein said PirB peptide fragment comprises a PirB peptide fragment selected from the group consisting of: SEQ ID NOs. 16-19.
   38. The composition of any of embodiment 35-36, wherein said PirB peptide fragment is coupled with a secretion signal domain through a linker domain.
   39. The composition of claim 38, wherein said secretion signal domain comprises a secretion signal according to SEQ ID NO. 14.
   40. The composition of claim 35, wherein said PirA/PirB complex comprises a dimer complex wherein PirA comprises a sequence according to SEQ ID NO. 1, and PirB comprises a sequence according to SEQ ID NO. 2.
   41. The composition of claim 35, wherein EMS is caused by a Vibrio sp.
   42. The composition of claim 35, wherein said aquatic organism comprises shrimp.
   43. A genetically modified microorganism expressing a heterologous polynucleotide operably linked to a promoter encoding a truncated PirB peptide of any of embodiment 35-40.
   44. A genetically modified microorganism expressing a heterologous polynucleotide operably linked to a promoter, wherein said heterologous polynucleotide encodes a peptide selected from the group consisting of: SEQ ID NOs. 3-4, and 5-11.
   45. The microorganism of any of embodiment 43-44, wherein the microorganism comprises a donor bacteria.
   46. The microorganism of claim 45, wherein said donor bacteria comprises Bacillus subtilis.
   47. A method of treating EMS in an aquatic animal comprising administering a therapeutically effective amount of a PirB peptide fragment of any of embodiment 35-40 to an aquatic animal that is infected by, or susceptible to infection by said EMS-causing bacterial pathogen, wherein said PirB peptide fragment competitively inhibits the formation of the PirA/PirB dimer complex.
   48. The method of claim 47, wherein said aquatic organism comprises shrimp.
   49. The method of claim 47, wherein administering comprises administering a therapeutically effective amount of a donor bacteria engineered to express a PirB peptide fragment of any of embodiment 35-40.
   50. The method of claim 47, wherein administering comprises administering a therapeutically effective amount of a donor bacteria engineered to express a PirB peptide fragment of any of embodiment 35-40, wherein said donor bacteria is incorporated into a treated feed or liquid inoculum.
   51. The method of any of embodiment 49-50, wherein the donor bacteria comprises a probiotic donor bacteria.
   52. The method of claim 51, wherein said probiotic donor bacteria comprises Bacillus subtilis.
   53. A method of treating Early Mortality Syndrome (EMS) in an aquatic organism comprising the steps of:
generating a donor microorganism to express a heterologous polynucleotide operably linked to a promoter encoding a PirB peptide fragment configured to competitively inhibit the formation of the PirA/PirB dimer complex produced by an EMS-causing bacterial pathogen;
introducing said genetically modified donor microorganism to a target host that is infected by, or susceptible to infection by said EMS-causing bacterial pathogen;
expressing said heterologous PirB peptide fragment and inhibiting the formation of the PirA/PirB dimer complex produced by an EMS-causing bacterial pathogen.
   54. The method of claim 53, wherein said PirB peptide fragment comprises a PirB peptide fragment encoding a portion of a binding interface with PirA.
   55. The method of any of embodiment 53-54, wherein said PirB peptide fragment comprises a PirB peptide fragment selected from the group consisting of: SEQ ID NOs. 16-19.
   56. The method of any of embodiment 53-54, wherein said PirB peptide fragment is coupled with a secretion signal domain through a linker domain.
   57. The method of claim 56, wherein said secretion signal domain comprises a secretion signal according to SEQ ID NO. 14.
   58. The method of claim 53, wherein said PirA/PirB complex comprises a dimer complex wherein PirA comprises a sequence according to SEQ ID NO. 1, and PirB comprises a sequence according to SEQ ID NO. 2.
   59. The method of claim 53, wherein said EMS-causing bacterial pathogen comprises a Vibrio sp.
   60. The method of claim 53, wherein said aquatic organism comprises shrimp.
   61. A genetically modified microorganism expressing a heterologous polynucleotide operably linked to a promoter encoding a PirB peptide fragment of any of embodiment 35-40.
   62. A genetically modified microorganism expressing a heterologous polynucleotide operably linked to a promoter, wherein said heterologous polynucleotide encodes a peptide selected from the group consisting of: SEQ ID NOs. 16-19.
   63. The bacteria of any of embodiment 61-62, wherein the microorganism comprises a donor bacteria.
   64. The bacteria of embodiment 63, wherein said donor bacteria comprises Bacillus subtilis.
   65. An isolated peptide selected from the group consisting of: SEQ ID NOs. 3-4, 5-11 and 16-19.
   66. An isolated nucleotide sequence encoding a peptide selected from the group consisting of: SEQ ID NOs. 3-4, 5-11 and 16-19.
   67. An isolated nucleotide sequence encoding a modified PirB peptide selected from the group consisting of: SEQ ID NOs. 12-13.
   68. An expression vector comprising a nucleotide sequence of any of embodiment 66-67, operably linked to a promoter.
   69. A microorganism transformed with the expression vector of embodiment 68.

Additional aspects of the invention will be evident from the detailed figures and descriptions below.

### BRIEF DESCRIPTION OF THE DRAWINGS

FIG. 1 shows PirA/PirB structural model (A) and truncated variants of Δ1-262 PirB^{Vp} PB-Sr (B), and F276S/A367T/P395Y mutational derivative of truncated variants of Δ1-262 PirB^{Vp} PB-Sr (C) having predicted enhanced affinity towards PirA.

FIG. 2 shows the construct design for the PB-Sr secreted peptide. (A) Map of plasmid designed for efficient expression of secreted variant of PB-Sr in both gram-positive and gram-negative bacteria. (B) Schematic drawing of secreted PB-Sr variant.

FIG. 3 shows expression pattern of PirA^{Vp}/PirB^{Vp}. Expression of pirA^{Vp}/pirB^{Vp} operon (A) is regulated by σS directed promoter (underlined, B) in the stationary phase of V. parahaemolyticus culture (C). PirA toxin is accumulated in the Vibrio cells in high concentrations during the bacterial growth (D).

FIG. 4 shows PB-Sr expression by B. subtilis BCG322(pAD-PB-Sr) decreases cytotoxicity of V. parahaemolyticus. Human cells cytotoxicity of V. parahaemolyticus supernatants is maximal in stationary phase of V. parahaemolyticus growth—bars. Co-growth of B. subtilis BCG322(pAD-PB-Sr) with V. parahaemolyticus decreases cytotoxicity. Average of the ratio of at least 3 biological. repeats is shown as dots with standard errors.

FIG. 5 shows feeding shrimp by BCG322-PB-Sr decreases shrimp mortality during the infection with V. parahaemolyticus. Positive control—shrimp fed by BCG322-pLuc. Negative control no V. parahaemolyticus infection.

FIG. 6 shows PirB derived peptide 214-WADNDSYNNANQDNVYDEVMGAR-236 (SEQ ID NO. 16) having high affinity to PirA as shown by flexible docking. Sample comparison of the conformational change for a PirB^{Vp}-based peptide (shown as dark gray surface) before (A) and after (B) flexible docking to PirA^{Vp} (shown as light gray cartoons).

FIG. 7 shows the structures of PirB and PirA. The regions thought to be involved, or not involved in the PirB/PirA interaction are colored blue (dark grey) and red light grey), respectively. The putative pore-forming domain in the N-terminal region that is thought to become exposed due to conformational changes after formation of the.

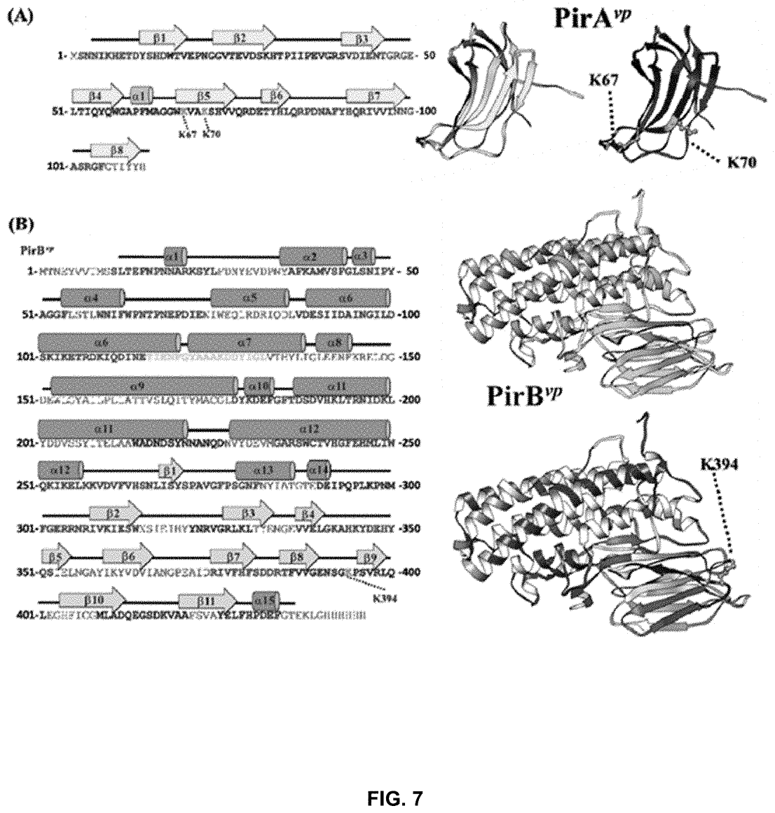

### DETAILED DESCRIPTION OF THE INVENTION

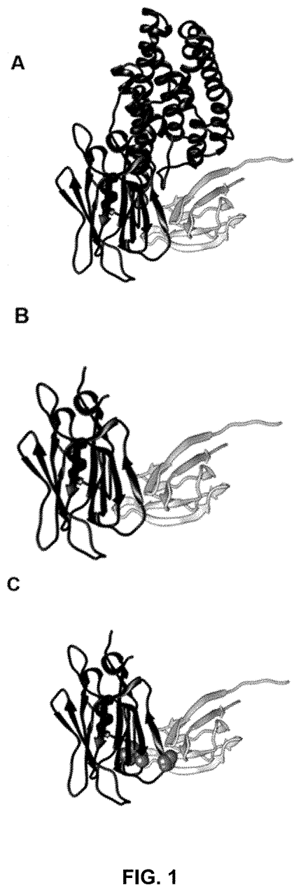

As noted above, EMS is one of the most devastating diseases in shrimp farming caused by pathogenic Vibrio sp., mainly by V. harveyi and V. parahaemolyticus. The death of infected shrimp happens as result of disruption of shrimp hepatopancreas epithelial cells by PirA/PirB toxins complexes. The presence of both PirA and PirB toxins and their interaction is necessary for toxicity. As demonstrated in FIG. 1A, analysis of protein structure revealed that PirB has 2 domains with different structural features. N-terminal domain contains the pore-forming element, responsible for disruption of host cells membrane; and C-terminal domain is involved in receptor binding and for interaction with PirA protein.

In one preferred embodiment, a composition of the invention may include a modified PirB toxin, and preferably a truncated PirB peptide encoding all or part of the C-terminal domain of PirB. In one preferred embodiment, a truncated PirB peptide may be a used as a therapeutic composition for the treatment of Early Mortality Syndrome (EMS) in an aquatic organism such as shrimp. In this embodiment, a truncated PirB peptide may include the deletion of the N-terminal domain that contains the pore-forming element, which may encompass residues 1-262. This truncated PirB Δ1-262 peptide (SEQ ID NO. 3) may act as a competitive inhibitor to PirB which may inactivate PirA/PirB toxin activation by competitive inhibitions of toxin-specific receptors on hepatopancreas cells, and/or by inhibits the formation of the PirA/PirB dimer complex.

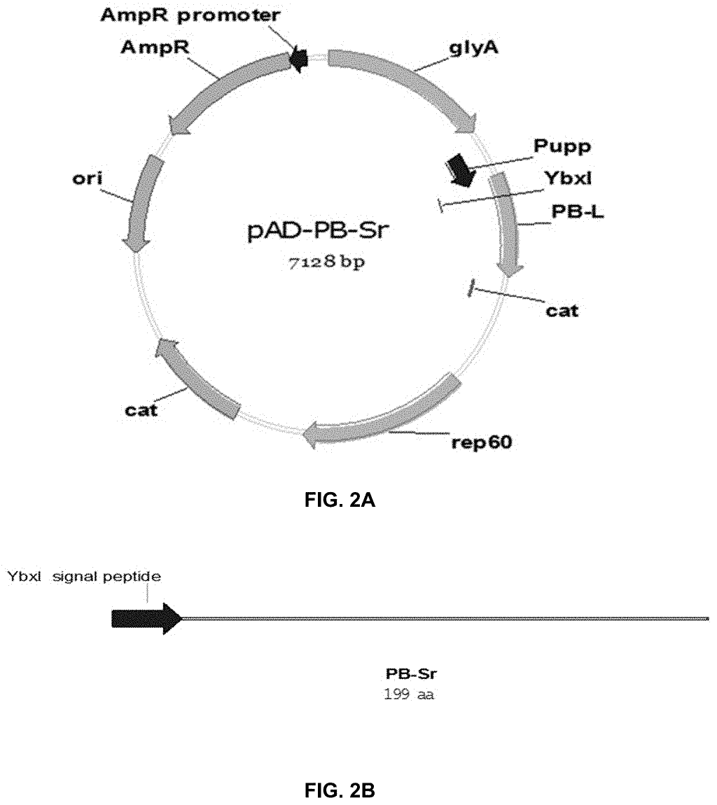

To help the modified PirB peptides of the invention better compete with wild type PirB (SEQ ID NO. 2) and bind to PirA (SEQ ID NO. 1) preventing active PirA/PirB binary toxin formation and its interactions to the cell membranes, it may be coupled with a secretion signal domain. For example, as shown in FIG. 2B, a truncated PirB Δ1-262 peptide (SEQ ID NO. 3) may be coupled with a secretion signal domain, directly, or through a linker peptide or other compound, such as a polyethylene glycol (PEG) linker. In a preferred embodiment, a truncated PirB Δ1-262 peptide of the invention may be coupled with an YbxI secretion signal (SEQ ID NO. 14) from Bacillus subtilis, forming a secretable truncated PirB Δ1-262 peptide (SEQ ID NO. 14) that, as detailed below, may be expressed in a donor bacteria and secreted into the extracellular environment where it can competitively inhibit PirB's ability to bind to PirA preventing active PirA/PirB binary toxin formation.

Specific point mutations may further be introduced that increase the binding affinity of a modified PirB peptide of the invention towards PirA thereby increasing its competitive inhibition of the formation of the PirA/PirB dimer complex. For example, a modified PirB peptide may include a truncated PirB peptide, and preferably a truncated PirB Δ1-262 peptide that may include one more point mutations at positions 276, 367 or 395 that increase the binding affinity of the truncated PirB Δ1-262 peptide towards PirA (SEQ ID NO. 1). In a preferred aspect, the truncated PirB Δ1-262 (SEQ ID NO. 3) peptide may include one more of the following point mutations selected from the group consisting of: F276S (SEQ ID NO. 5), A367T (SEQ ID NO. 6), P395Y (SEQ ID NO. 7), or any combination thereof. In another preferred embodiment, the truncated PirB Δ1-262 peptide may include a combination of substitution mutations that increase the binding affinity of the truncated PirB Δ1-262 peptide towards PirA (SEQ ID NO. 1) selected from the group consisting of: F276S/A367T (SEQ ID NO. 8), F276S/P395Y (SEQ ID NO. 9), A367T/P395Y (SEQ ID NO. 10), and F276S/A367T/P395Y (SEQ ID NO. 11).

The inventive technology further includes methods of treating EMS in an aquatic animal, and preferably shrimp, which may include administering a therapeutically effective amount of a modified PirB peptide to an aquatic animal that is infected by, or susceptible to infection by an EMS-causing bacterial pathogen. Preferred embodiments may include administering a therapeutically effective amount of a truncated PirB Δ1-262 peptide according to SEQ ID NOs. 3-11, wherein the truncated PirB peptide competitively inhibits the formation of the PirA/PirB dimer complex. A truncated PirB peptide may be administered directly to an aquatic animal, such as a shrimp, for example by injection. In an alternative embodiment, truncated PirB peptide of the invention may be administered by donor bacteria engineered to express a truncated PirB. For example, a bacterial strain may be identified that is symbiotic, endosymbiotic, or probiotic (generally being referred to as “probiotic”) with a target host, which may preferably include an aquatic animal host, and more preferably a shrimp host produced in aquaculture. An exemplary endosymbiotic bacteria may include E. coli, or Enterobacter strain Agl identified by Sayre et al., in PCT/US2018/045687, or Bacillus subtilis strain (BG322) identified by Sayre et al., in PCT/US2018/045687, all of which being incorporated herein by reference.

These probiotic bacteria may be genetically modified to include a nucleotide sequence, operably linked to a promoter, which expresses a truncated PirB peptide, such as those according to SEQ ID NOs. 3-11. The genetically modified probiotic bacteria expressing a truncated PirB peptide may preferably be administered to an aquatic animal, for example through a treated feed or liquid inoculum method—such feeds and inoculums supplemented with bacteria or bacterial spores from probiotic bacteria being readily known by those of ordinary skill in the art. Even where high levels of Vibrio infection are present in an aquaculture environment, administering a therapeutically effective amount of the genetically modified probiotic bacteria expressing a truncated PirB peptide, may persist in the environment and provide continuing local protection from Vibrio toxins and obviating the need for repeated administrations.

The invention may specifically include a method of treating Early Mortality Syndrome (EMS) in an aquatic organism comprising the steps of: generating a donor microorganism to express a heterologous polynucleotide operably linked to a promoter encoding a modified PirB peptide, and preferably truncated PirB peptide according to SEQ ID NOs. 3-11, configured to competitively inhibit the formation of the PirA/PirB dimer complex produced by an EMS-causing bacterial pathogen. This donor microorganism, which may preferably include a shrimp probiotic strain of bacteria such as B. subtilis, may be introduced to a target host, such as shrimp in an aquaculture environment, that is infected by, or susceptible to infection by said EMS-causing bacterial pathogen. The donor microorganism may colonize the shrimp in this embodiment and express said heterologous modified PirB peptide, and preferably truncated PirB peptide according to SEQ ID NOs. 3-11 which may further be secreted out of the cell where it may inhibit the formation of the PirA/PirB dimer complex produced by an EMS-causing bacterial pathogen in the target host.

In one preferred embodiment, a composition of the invention may include a modified PirB peptide, and preferably a PirB peptide fragment encoding all or part of a binding interface domain with PirA. In one preferred embodiment, a PirB peptide fragment may be used as a therapeutic composition for the treatment of Early Mortality Syndrome (EMS) in an aquatic organisms such as shrimp. In this embodiment, a PirB peptide fragment may include fragments of PirB located between residues 214 and 401 that may interact with PirA. Such PirB peptide fragment may act as a competitive inhibitor to PirB which may inactivate PirA/PirB toxin activation by competitive inhibitions of toxin-specific receptors on hepatopancreas cells, and/or by inhibits the formation of the PirA/PirB dimer complex. In one specific embodiment, a PirB peptide fragment may include a peptide selected from the group consisting of SEQ ID NOs. 16-19. In an optionally embodiment, PirB peptide fragment may be coupled with a secretion signal domain, directly, or through a linker peptide or other compound, such as a polyethylene glycol (PEG) linker. In a preferred embodiment, a PirB peptide fragment of the invention may be coupled with a include an YbxI secretion signal (SEQ ID NO. 14) from Bacillus subtilis, forming a secretable PirB peptide fragment that, as detailed below, may be expressed in a donor bacteria and secreted into the extracellular environment where it can competitively inhibit PirB's ability to bind to PirA preventing active PirA/PirB binary toxin formation.

The inventive technology further includes methods of treating EMS in an aquatic animal, and preferably shrimp, which may include administering a therapeutically effective amount of a modified PirB peptide, and preferably a PirB peptide fragment, to an aquatic animal that is infected by, or susceptible to infection by an EMS-causing bacterial pathogen. Preferred embodiments may include administering a therapeutically effective amount of a PirB peptide fragment peptide according to SEQ ID NOs. 16-19, wherein the PirB peptide fragment competitively inhibits the formation of the PirA/PirB dimer complex.

A PirB peptide fragment may be administered directly to an aquatic animal, such as a shrimp, for example by injection. In an alternative embodiment, a PirB peptide fragment of the invention may be administered by a donor bacteria engineered to express a PirB peptide fragment. For example, a bacterial strain may be identified that is probiotic with the aquatic animal, such as shrimp. These probiotic bacteria may be genetically modified to include a nucleotide sequence, operably linked to a promoter, which expresses a PirB peptide fragment, such as those according to SEQ ID NOs. 16-19. The genetically modified probiotic bacteria expressing a PirB peptide fragment may preferably be administered to an aquatic animal, for example through a treated feed or liquid inoculum method—such feeds and inoculums supplemented with bacteria or bacterial spores from probiotic bacteria being readily known by those of ordinary skill in the art. Even where high levels of Vibrio infection are present in an aquaculture environment, administering a therapeutically effective amount of the genetically modified probiotic bacteria expressing a PirB peptide fragment, may persist in the environment and provide continuing local protection from Vibrio toxins and obviating the need for repeated administrations.

The invention may specifically include a method of treating Early Mortality Syndrome (EMS) in an aquatic organism comprising the steps of: generating a donor microorganism to express a heterologous polynucleotide operably linked to a promoter encoding a modified PirB peptide, and preferably a PirB peptide fragment according to SEQ ID NOs. 16-19, configured to competitively inhibit the formation of the PirA/PirB dimer complex produced by an EMS-causing bacterial pathogen. This donor microorganism, which may preferably include a shrimp probiotic strain of bacteria such as B. subtilis, may be introduced to a target host, such as shrimp in an aquaculture environment, that is infected by, or susceptible to infection by said EMS-causing bacterial pathogen. The donor microorganism may colonize the shrimp in this embodiment and express said heterologous modified PirB peptide, and preferably a PirB peptide fragment according to SEQ ID NOs. 16-19 which may optionally be secreted or transported out of the bacterial cell, for example by outer membrane vesicles (OMVs) where it may inhibit the formation of the PirA/PirB dimer complex produced by an EMS-causing bacterial pathogen in the target host.

The term “aquaculture” as used herein includes the cultivation of aquatic organisms under controlled conditions.

The term “aquatic organism” and/or “aquatic animal” as used herein includes organisms grown in water, either fresh or saltwater. Aquatic organisms/animals includes vertebrates, invertebrates, arthropods, fish, mollusks, including, shrimp (e.g., penaeid shrimp, Penaeus esculentu, Penaeus setiferus, Penaeus stylirostris, Penaeus occidentalis, Penaeus japonicus, Penaeus vannamei, Penaeus monodon, Penaeus chinensis, Penaeus aztecus, Penaeus duorarum, Penaeus indicus, and Penaeus merguiensis, Penaeus calif orniensis, Penaeus semisulcatus, Penaeus monodon, brine shrimp, freshwater shrimp, etc), crabs, oysters, scallop, prawn clams, cartilaginous fish (e.g., sea bream, trout, bass, striped bass, tilapia, catfish, salmonids, carp, catfish, yellowtail, carp zebrafish, red drum, etc), crustaceans, among others. Shrimp include shrimp raised in aquaculture as well.

The term “probiotic” refers to a microorganism, such as bacteria, that may colonize a host and provide a benefit. The term “probiotic” also refers to a microorganism, such as bacteria, that may colonize a host for a sufficient length of time to deliver a therapeutic or effective amount of a truncated toxin peptide. A probiotic may include enteric, symbiotic, and endosymbiotic bacteria, or naturally occurring flora that may permanently to temporarily colonize an animal, such as an aquatic organism, and preferably shrimp. Specific examples of bacterial vectors include bacteria (e.g., cocci and rods), filamentous algae and detritus. Specific embodiments of transformable bacterial vectors cells that may be endogenous through all life cycles of the host may include all those listed herein. Additional embodiments may include one or more additional bacterial strains.

As used herein, the term modified may include a peptide that has one or more amino acid residues mutated or removed. In other embodiments, a modified peptide may include a truncated peptide that may include a peptide that has one or more amino acid residues that correspond to a specific domain that have further been removed or mutated so as to generate a loss of function in that domain. In other embodiments, a modified peptide may include a peptide fragment that may include a discrete portion of a peptide sequence that may act as a competitive inhibitor with the wildtype peptide to which it corresponds.

The term “operon” refers to a unit made up of linked genes.

As used herein, Vibrio is a genus of Gram-negative, facultative anaerobic bacteria possessing a curved-rod shape, with Vibrio sp. indicating a species within the genus Vibrio. In some embodiments, Vibrio sp. can comprise any one or more of the following Vibrio species, and in all possible combinations: adaptatus, aerogenes, aestivus, aestuarianus, agarivorans, albensis, alfacsensis, alginolyticus, anguillarum, areninigrae, artabrorum, atlanticus, atypicus, azureus, brasiliensis, bubulus, calviensis, campbellii, casei, chagasii, cholera, cincinnatiensis, coralliilyticus, crassostreae, cyclitrophicus, diabolicus, diazotrophicus, ezurae, fischeri, fluvialis, fortis, furnissii, gallicus, gazo genes, gigantis, halioticoli, harveyi, hepatarius, hippocampi, hispanicus, hollisae, ichthyoenteri, indicus, kanaloae, lentus, litoralis, logei, mediterranei, metschnikovii, mimicus, mytili, natriegens, navarrensis, neonates, neptunius, nereis, nigripulchritudo, ordalii, orientalis, pacinii, parahaemolyticus, pectenicida, penaeicida, pomeroyi, ponticus, proteolyticus, rotiferianus, ruber, rumoiensis, salmonicida, scophthalmi, splendidus, superstes, tapetis, tasmaniensis, tubiashii, vulnificus, wodanis, and xuii.

As used herein, the phrase “host” or “target host” refers to an organism or population carrying a disease-causing pathogen, or an organism or population that is susceptible to a disease-causing pathogen. A “host” or “target host” may further include an organism or population capable of carrying a disease-causing pathogen.

As used herein, the terms “controlling” and/or “bio-control” refer to reducing and/or regulating pathogen/disease progression and/or transmission.

As used herein, the phrase “feed” refers to animal consumable material introduced as part of the feeding regimen or applied directly to the water in the case of aquatic animals. A “treated feed” refers to a feed treated with, or containing a bacteria or bacterial spore, configured to express a modified toxin peptide, such as a modified PirB peptide as generally described herein. A “feed” may also be an aquatic animal, or a shrimp culture pond/aquaculture inoculum.

The term “nucleic acid” as used herein, refers to a polymer of ribonucleotides or deoxyribonucleotides. Typically, “nucleic acid or “nucleic acid agent” polymers occur in either single or double-stranded form but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

The term “recombinant” when used with reference, e.g., to a cell, or nucleic acid, protein, or vector, indicates that the cell, organism, nucleic acid, protein or vector, has been modified by the introduction of a heterologous nucleic acid or protein or the alteration of a native nucleic acid or protein, or that the cell is derived from a cell so modified. Thus, for example, recombinant cells may express genes that are not found within the native (non-recombinant or wild-type) form of the cell or express native genes that are otherwise abnormally expressed, over-expressed, under expressed or not expressed at all.

The terms “genetically modified,” “bio-transformed,” “transgenic”, “transformed”, “transformation”, and “transfection” are similar in meaning to “recombinant”. “Transformation”, “transgenic”, and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur, the organism or cell may need to be kept under certain conditions. Host organisms or cells containing the recombinant polynucleotide can be referred to as “transgenic” or “transformed” organisms or cells or simply as “transformants”, as well as recombinant organisms or cells.

The term “vector” refers to some means by which DNA, RNA, a protein, or polypeptide can be introduced into a host. The polynucleotides, protein, and polypeptide which are to be introduced into a host can be therapeutic or prophylactic in nature; can encode, or be an antigen; can be regulatory in nature; etc. There are various types of vectors including virus, plasmid, bacteriophages, cosmids, and bacteria. An “expression vector” is a nucleic acid capable of replicating in a selected host cell or organism. An expression vector can replicate as an autonomous structure, or alternatively can integrate, in whole or in part, into the host cell chromosomes or the nucleic acids of an organelle, or it may be used as a shuttle for delivering foreign DNA to cells, and thus replicate along with the host cell genome. Thus, expression vectors are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector of this invention. The use of the cassette assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is “operably linked” to an expression control sequence(s) (e.g., a promoter and, optionally, an enhancer) when the expression control sequence controls and regulates the transcription and/or translation of that polynucleotide sequence. As used herein, the phrase “gene product” refers to an RNA molecule or a protein. Moreover, the term “gene” may sometime refer to the genetic sequence, the transcribed and possibly modified mRNA of that gene, or the translated protein of that mRNA. As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. Examples of suitable promoters for gene suppressing cassettes include, but are not limited to, Pupp, T7 promoter, bla promoter, U6 promoter, pol II promoter, Ell promoter, and CMV promoter and the like. Optionally, each of the promoter sequences of the gene promoting cassettes and the gene suppressing cassettes can be inducible and/or tissue-specific.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The terms “peptide”, “polypeptide”, and “protein” are used to refer to polymers of amino acid residues. These terms are specifically intended to cover naturally occurring biomolecules, as well as those that are recombinantly or synthetically produced, for example by solid phase synthesis.

According to a specific embodiment, the vector for the heterologous truncated toxin protein, such as a modified PirB peptide, or donor is bacteria. In other embodiments, the donor is an algae cell. Various algae species can be used in accordance with the teachings of the invention since they are a significant part of the diet for many kinds of hosts that feed opportunistically on microorganisms as well as on small aquatic animals such as rotifers. Examples of algae that can be used in accordance with the present teachings include, but are not limited to, blue-green algae as well as green algae. Specifically, Actinastrum hantzschii, Ankistrodesmus falcatus, Ankistrodesmus spiralis, Aphanochaete elegans, Chlamydomonas sp., Chlorella ellipsoidea, Chlorella pyrenoidosa, Chlorella variegate, Chlorococcum hypnosporum, Chodatella brevispina, Closterium acerosum, Closteriopsis acicularis, Coccochloris peniocystis, Crucigenia lauterbomii, Crucigenia tetrapedia, Coronastrum ellipsoideum, Cosmarium botrytis, Desmidium swartzii, Eudorina elegans, Gloeocystis gigas, Golenkinia minutissima, Gonium multicoccum, Nannochloris oculata, Oocystis marssonii, Oocystis minuta, Oocystis pusilla, Palmella texensis, Pandorina morum, Paulschulzia pseudovolvox, Pediastrum clathratum, Pediastrum duplex, Pediastrum simplex, Planktosphaeria gelatinosa, Polyedriopsis spinulosa, Pseudococcomyxa adhaerans, Quadrigula closterioides, Radiococcus nimbatus, Scenedesmus basiliensis, Spirogyra pratensis, Staurastrum gladiosum, Tetraedron bitridens, Trochiscia hystrix. Anabaena catenula, Anabaena spiroides, Chroococcus turgidus, Cylindrospermum licheniforme, Bucapsis sp. (U. Texas No. 1519), Lyngbya spiralis, Microcystis aeruginosa, Nodularia spumigena, Nostoc linckia, Oscillatoria lutea, Phormidiumfaveolarum, Spinilina platensis. A donor microorganism may also be a yeast cell.

In a further embodiment, a composition including genetically modified bacteria configured to express a truncated toxin peptide may be formulated as a “treated feed” which may include a water dispersible granule or powder that may further be configured to be dispersed into the environment. In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner. Alternatively, or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules, or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like). The formulations or compositions containing genetically modified bacteria may include spreader-sticker adjuvants, stabilizing agents, other pesticidal additives, or surfactants. Liquid formulations may be employed as foams, suspensions, emulsifiable concentrates, or the like. The ingredients may include biological agents, surfactants, emulsifiers, dispersants, or polymers.

Compositions of the invention, which may include genetically modified donor bacteria expressing heterologous modified toxin proteins, can be used for the bio-control of pathogens in an animal or other host. Such an application comprises administering to a host an effective amount of the composition which expresses from the donor sufficient heterologous modified toxin proteins, such as a modified PirB peptide, or a combination of both, that may be transported out of the donor and taken-up by the target pathogen, thus interfering with binding and/or activity or the toxin, for example by inhibiting the PirB/PirA dimer complex and thereby controlling the pathogen and/or pathogen's disease causing effects on the host.

Compositions of the invention can be used for the control of pathogen gene expression and its effects described herein, in vivo. Such an application comprises administering to target host, such as shrimp, an effective amount of the composition which inhibits the binding or activity of the pathogen created toxin carried by the host, reducing or eliminating the disease state in the host. Thus, regardless of the method of application, the amount of the genetically modified symbiotic donor bacteria expressing heterologous truncated toxin proteins that may be applied at an therapeutically effective amount to inhibit its effects, will vary depending on factors such as, for example, the specific host to be controlled, the type of pathogen, in some instances the water source to be treated, the environmental conditions, and the method, rate, and quantity of application of the composition. The concentration of the composition that is used for environmental, systemic, or foliar application will vary widely depending upon the nature of the particular formulation, means of application, environmental conditions, and degree of biocidal activity.

According to some embodiments, a heterologous modified toxin protein, such as a modified PirB peptide is provided in therapeutically effective amounts to reduce or inhibit the toxins pathogenic activity. As used herein “an effective amount” or a “therapeutically effective amount” refers to an amount of donor bacteria producing a heterologous truncated toxin protein which is sufficient to inhibit the activity or pathogenic action of the target toxin, by at least 5%, 10% 20%, 30%, 40%, 50%, or more, say 60%, 70%, 80%, 90%, or even up to 100%. All ranges include the ranges in between those specifically stated.

As used herein, the term “gene” or “polynucleotide” refers to a single nucleotide or a polymer of nucleic acid residues of any length. The polynucleotide may contain deoxyribonucleotides, ribonucleotides, and/or their analogs, and may be double-stranded or single stranded. A polynucleotide can comprise modified nucleic acids (e.g., methylated), nucleic acid analogs or non-naturally occurring nucleic acids, and can be interrupted by non-nucleic acid residues. For example, a polynucleotide includes a gene, a gene fragment, cDNA, isolated DNA, mRNA, tRNA, rRNA, and isolated RNA of any sequence, recombinant polynucleotides, primers, probes, plasmids, and vectors. Included within the definition, are nucleic acid polymers that have been modified, whether naturally or by intervention. Additionally, reference to a nucleotide sequence also encompasses and specifically incorporates it corresponding amino acid sequence and vice versa.

As used herein the terms “approximately” or “about” refer to ±10%>. Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

The terms “comprises”, “comprising”, “includes”, “including”, “having” and their conjugates mean “including but not limited to.” The term “consisting of means “including and limited to”. The term “consisting essentially of means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references, unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “system” and/or “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing, or reversing the progression of a condition, substantially ameliorating clinical or aesthetical symptoms of a condition or substantially preventing the appearance of clinical or aesthetical symptoms of a condition.

As used herein, “symbiotic” or “symbionts” generally refers to a bacterium that is a symbiont of a host. It may also include bacteria that persist throughout the life-cycle of a host, either internally or externally, and may further be passed horizontally to the offspring or eggs of a host. Symbionts can also include bacteria that colonize outside of host's cells and even in the tissue, lymph, or secretions of the host. Endosymbionts generally refers to a subgroup of internal symbionts.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

As used herein, “inhibits,” “inhibition” refers to the decrease in protein interaction relative to the normal wild type level, or control level. Inhibition may result in a decrease in protein binding, such as PirB and PirA binding in response to the inhibition by a modified PirB peptide of the invention of the invention by less than 10%, 10%, 15%, 20%, 25%, 30%, 35%, 40%, 45%, 50%, 55%, 60%, 65%, 70%, 75%, 80%, 85%, 90%, 95% or 100%.

This invention utilizes routine techniques in the field of molecular biology. Basic texts disclosing the general methods of use in this invention include Green and Sambrook, 4th ed. 2012, Cold Spring Harbor Laboratory; Kriegler, Gene Transfer and Expression: A Laboratory Manual (1993); and Ausubel et al., eds., Current Protocols in Molecular Biology, 1994-current, John Wiley & Sons. Unless otherwise noted, technical terms are used according to conventional usage. Definitions of common terms in molecular biology maybe found in e.g., Benjamin Lewin, Genes IX, published by Oxford University Press, 2007 (ISBN 0763740632); Krebs, et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Design of Construct Expressing Truncated PirB^{Vp}

To prevent formation of active PirA^{Vp}/PirB^{Vp} binary toxin (see model at FIG. 1A) the present inventors employed B. subtilis expressing Δ1-262 PirB^{Vp} truncated variant. (FIG. 1B; SEQ ID NO. 3, also referred to as PirB^{Vp}-Sr). To design the PirB^{Vp}-Sr, we used a published model of the PirA^{Vp}/PirB^{Vp} dimer to select optimal truncation sites. Truncation should inhibit PirB^{Vp} interaction with the cell membrane and toxin pore formation but should not affect PirA^{Vp} binding. The PirB^{Vp}-Sr amino acid sequence was engineered to include an N-terminal secretion peptide, in this embodiment YhxI to allow release of the PirB^{Vp}-Sr-YbxI into the bacterial surroundings (See FIG. 2, amino acid sequence SEQ ID NO. 4, DNA sequence SEQ ID NO. 13). In this configuration, the engineered PirB^{Vp}-Sr may be expressed in B. subtilis and secreted into the bacterial surroundings where it may interact with V. parahaemolyticus expressed PirA^{Vp} thus alleviating PirA^{Vp}/PirB^{Vp} cytotoxicity and shrimp EMS-induced disease.

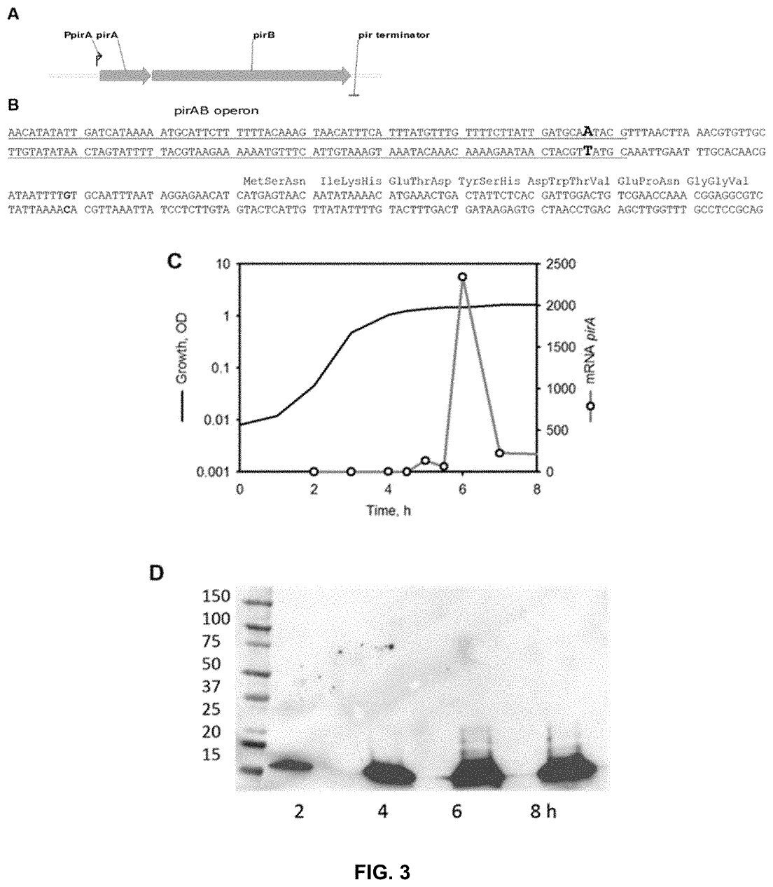

In order to select a proper promoter, the present inventors studied the expression patterns of the pirA^{Vp}/pirB^{Vp} operon (FIG. 3A). A pirA^{Vp}/pirB^{Vp} transcription starting point was established with a predicted existence of a strong σ^{S} directed promoter driving pirA^{Vp}/pirB^{Vp} transcription (FIG. 3B). It is known that σ^{S} directed transcription of the genes occurs in the late phase of bacterial growth when nutrients are limited. In agreement with this, the present inventors found that expression of pirA^{Vp}/pirB^{Vp} was dramatically increased in the stationary phase of Vibrio growth when the concentration of bacterial cells are high (FIG. 3C). Accordingly, PirA protein is accumulated in the Vibrio cells in very high amounts during stationary phase (FIG. 3D). In order to counteract toxicity, expression of PirB^{Vp} (PB-Sr) antitoxin was engineered under a strong Pupp promoter of plasmid pAD43-25 that is active during all phases of Bacillus growth. DNA fragment encoding PirB^{Vp}-Sr-YbxI sequence (SEQ ID NO. 4) was ordered and cloned into pAD43-25 B. subtilis-E. coli shuttle vector. A final expression construct pAD-PB-Sr was transformed into the competent cells of B. subtilis BCG322 (See FIG. 2, Table 1).

### Example 2: PirB^{Vp}-Sr Expression by Bacillus Decreases Cytotoxicity of V. parahaemolyticus

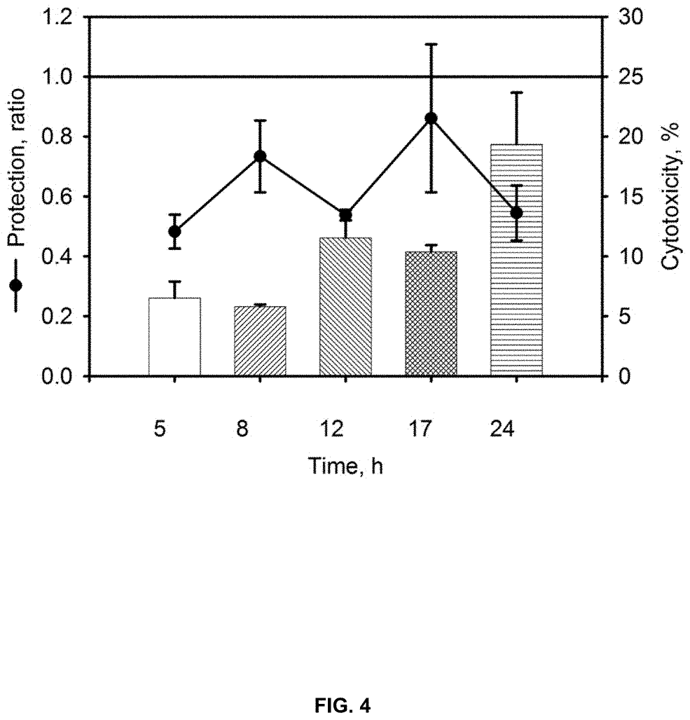

As noted above, in order to act, PB-Sr must be secreted into the media to compete with wild-type PirB^{Vp} and bind to PirA^{Vp} preventing active PirA^{Vp}/PirB^{Vp} binary toxin formation and its interactions to the cell membranes. All of these events are predicted to significantly decrease PirAB^{Vp} cytotoxicity to shrimp cells. Since shrimp cell culture is not available the present inventors studied PirAB^{Vp} cytotoxicity using HeLa human cell culture. A mixed culture of BCG322 (pAD-PB-Sr) and V. parahaemolyticus was prepared, with samples taken at certain time points which were then incubates in bacterial cell-free cultural media with HeLa cell. Cytotoxicity was measured by release of lactate dehydrogenase (LDH) from the disrupted cells into the surrounding. In this example, BCG322 (pAD-luc) served as control strain. As shown in FIG. 4, overall cytotoxicity was not found to be high, which may be expected since human cells are not natural target for PirAB^{Vp}. However, cytotoxicity was readily detected in this assay. Moreover, co-growth of BCG322 (pAD-PB-Sr) with V. parahaemolyticus decreased cytotoxicity of cell-free cultural media two-folds compared with control demonstrating that expression of truncated PirB^{Vp} coupled with an YbxI secretion signal peptide decreases Vibrio cytotoxicity.

### Example 3: Bacillus-Directed PB-S Expression Decreases Shrimp Mortality During the Experimental Infection with V. parahaemolyticus

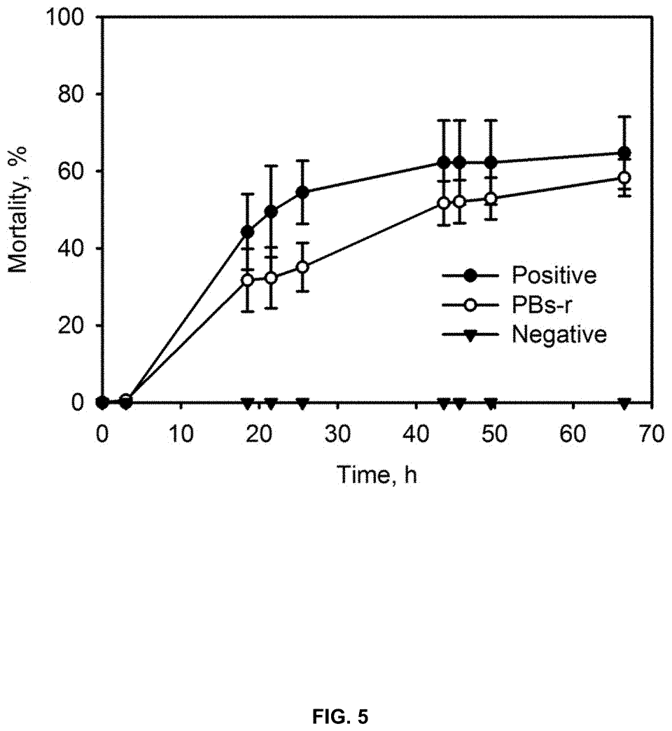

To demonstrate that decreased cytotoxicity may provide prophylactic protection against the EMS-causing pathogen V. parahaemolyticus, shrimp were fed BCG322 (pAD-PB-Sr) and control strain BCG322 (pAD-luc) for three days to allow bacterial colonization in the shrimp intestines. Based on the lack of mortality or other observable pathologies, it was determined that BCG322 (pAD-PB-Sr) was safe to be used as shrimp food supplement. As shown in FIG. 5, sample shrimp populations were challenged by V. parahaemolyticus and their mortality was scored. After 24 h post-challenge, shrimp mortality was reduced by 2× in the shrimp groups that were fed by BCG322 (pAD-PB-Sr) demonstrating that inactivation of PirAB^{Vp} toxin by competition with truncated PirB^{Vp} could be the viable strategy to provide shrimp prophylactic protection from PirAB^{Vp}-induced EMS infections.

### Example 3: Computational Identification of Mutants in the PirB^{Vp}-Δ1-262 Truncation Mutant

As shown in Table 1 below, affinity of PirAB^{Vp} interactions is low. On the one hand, it renders the present competitive inhibition strategy easier—low affinity complexes are easily disrupted by competitors. On the other hand, if the affinity of a competitive inhibitor, such as PB-Sr is low too, the system may not be sufficiently robust to provide adequate competitive binding profiles. To improve affinity of the target “competitor” PB-Sr molecule, the present inventors performed in silico—directed rational design to identify one or more mutations that would increase PB-Sr binding affinity towards PirA.

As noted earlier, the PirB^{Vp}-Δ1-262 truncation mutant includes residues 263-438 that form the mostly β-sheet domain that in turn comprises the primary binding interface with PirA^{Vp} (FIG. 1B). Three main contact regions were identified on the interfacial surface of PirB^{Vp}-Δ1-262. For each of these contact regions, single mutations were then selected based on predictions that these have minimal impact on the folding free energy of the domain. The single mutations were then ranked based on predicted binding affinity to PirA^{Vp}. The top single mutant (F276S, A367T, and P395Y) in each contact region is given in Table 2 below along with their predicted PirA^{Vp}-binding affinities. Notably, the three single mutants have a predicted binding affinity that is around 4-5× stronger than for the unmodified truncation mutant. The predicted binding affinities for the three double mutant and one triple mutant combinations are also provided in Table 2 and indicate that the point mutations have combinatorial effects. In particular, the F276S/A367T/P395Y triple mutant of PirB^{Vp}-Δ1-262 is predicted to have stronger binding to PirA^{Vp} by around two orders of magnitude (compared with full WT PirB^{Vp}), making it a very effective predicted competitor against formation of the full PirA^{Vp}/PirB^{Vp} complex (FIG. 1C).

### Example 4: Computational Design of PirB^{Vp}-Based Peptide Competitors

Small peptides can be a valuable tool to disrupt protein-protein interactions, They may be easily synthesized in high amounts by bacterial cells and could be designed as highly active drugs. In order to select and evolve highly active peptide therapeutic compounds, several peptides from PirB^{Vp} that are part of its binding interface with PirA^{Vp} were selected for further analysis (See Table 3). The predicted binding affinities for each of these PirB^{Vp}-based peptides was identified after performing flexible docking which are also provided in Table 3. The present inventors opted to perform flexible backbone docking of each PirB^{Vp}-based peptide to PirA^{Vp} prior to binding affinity estimation, since derived peptides may adopt different conformations when not part of the full protein.

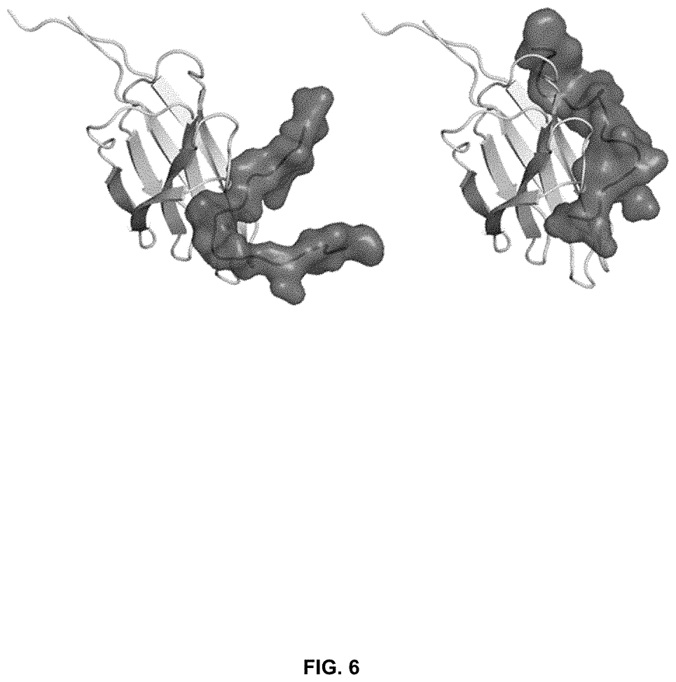

As shown in FIG. 6, the conformation of one of the peptides from the protein structure (left) is compared with the predicted conformation after flexible backbone docking (right). Note that the docking predicts a conformational change in the peptide that leads to a more extensive interaction interface with PirA^{Vp}. From the peptides in Table 3, peptide 214-WADNDSYNNANQDNVYDEVMGAR-236 (SEQ ID NO. 16) is predicted to have stronger binding to PirA^{Vp} by around two orders of magnitude (compared with full WT PirB^{Vp}), making it a predicted competitor against formation of the full PirA^{Vp}/PirB^{Vp} complex.

### Example 5: Materials and Methods

Rational design of PirB^{Vp}-Tr competitor: As shown in FIG. 1A, published structural models of the PirA^{Vp}/PirB^{Vp} dimer complex was used as the basis for the rational design of PirB^{Vp}-Tr variants. This dimer model was built using experimentally-constrained docking between the crystal structures of PirA^{Vp} (SEQ ID NO. 1), and PirB^{Vp} (SEQ ID NO. 2), For PirB^{Vp}, the α-helical domain from residues 1-262 comprises the pore-forming region while the mostly β-sheet domain from residues 263-438 contains the interaction interface with PirA^{Vp}. Based on this information, the present inventors used a truncation mutant PirB^{Vp}-Δ1-262 (amino acid sequence SEQ ID NO. 3; DNA sequence SEQ ID NO. 12) that includes only the PirB^{Vp}-interacting domain. After replacing the full PirB^{Vp} with the PirB^{Vp}-Δ1-262 mutant in the dimer model, protein-protein interface residues between PirA^{Vp} and PirB^{Vp}-Δ1-262 were identified using the online server for the CPORT algorithm. This approach found three main contact regions in the interfacial surface between both proteins. A combination of predictions from FoldX (for estimating folding free energies) and PRODIGY (for estimating protein-protein binding affinities) was then used to identify mutations at these three contact regions that would enhance the PirA^{Vp}-binding affinity of PirB^{Vp}-Δ1-262 while not impacting the stability of its fold.

Rational design of PirB^{Vp}-based peptide competitors: Several peptide sequences were selected from PirB^{Vp} that were involved in the binding interface with PirA^{Vp}. As peptides are not necessarily expected to maintain the same conformation as found in the proteins that they are derived from, the CABS-dock algorithm was used to perform flexible docking of each peptide onto the PirA^{Vp} binding interface. This approach allows for enhanced sampling of backbone conformation for the PirB^{Vp}-derived peptides, while the backbone of PirA^{Vp} was kept restrained and sampling of only side chain rotamers was permitted. Binding affinities for the top-ranked protein-peptide complexes were estimated using PRODIGY.

Vibrio parahaemolyticus AHPND strain: The target Vibrio strain was isolated from shrimp farms during an AHPND outbreak in Mexico. V. parathaemolyticus was grown in LBS (LB media (BD) supplied with 2.5% NaCl and incubated for 28 h at 30° C. (200 rpm), Bacterial DNA was obtained using the DNeasy Blood&Tissue kit (Qiagen). Presence of V. parahaemolyticus AHPND causing plasmid with pirA/pirB operon was confirmed by full genome sequencing and by PCR using the AP1 and AP2 primers.

Construct design: Bacterial strains and plasmids are listed in Table 1 below. PB-Sr protein and DNA sequences are shown below. pAD-PB-Sr plasmid expressing PirB^{Vp}-Δ1-262 under control of strong B. cereus promoter Pupp was ordered via Genscript. For the expressing plasmid, pAD43-25 was used as vector backbone.

PirA^{Vp}/PirB^{Vp} expression pattern: To determine total RNA of V. parahaemolyticus, cells was isolated using RNeasy Plus Mini kit (Qiagen) from samples taken at various time points of bacteria growth curve. RT-qPCR was performed using Luna® Universal One-Step RT-qPCR Kit (NEB) kit with AP1 and AP2 oligonucleotides.

Mapping of PpirA^{Vp} promoter: The present inventors performed 5′RACE (Rapid amplification of cDNA ends) assay on total RNA extracted overnight culture of V. parahaemolyticus using a 5′RACE System kit (Invitrogen). Reverse transcription was performed with the Superscript II enzyme using GSP1 primer, which either fell within the pirA^{Vp} or pirB^{Vp} genes. The cDNAs were purified on SNAP column and a polyC tail was added with Terminal deoxynucleotidyl transferase. Then, PCR was performed using a primer hybridizing with the polyC tail and pir-specific primers. PCR amplification products purified with PCR purification kit. The pirA^{Vp}/pirB^{Vp} operon transcription starting point determined by DNA sequencing.

PirA antibody production: Antibodies were ordered via. Genscript to peptide antigen, fragment of pirA CVQRDETYHLQRPDN (SEQ ID NO. 20) GenScript used its proprietary OptimumAntigen™ design tool and proprietary adjuvant. A cysteine is automatically added to the N terminus of the peptide to conjugate to KLH.

SDS-PAGE and Western blotting. Bacterial supernatants and crude extracts of V. parahaemolyticus cells were separated on 15% SDS-PAGE. For Western blot analysis, the samples resolved by SDS-PAGE were transferred onto nitrocellulose membranes using a Transblot apparatus (BioRad). Nitrocellulose membranes were incubated in 5% blocking solution for 10 min and treated with anti-PirA antibodies for 4 h. Anti-rabbit HPR secondary antibody were used to visualization.

Shrimp mortality. Pacific white shrimp (Litopenaeus vannamei) post-larvae were obtained and maintained at the Zeigler holding facilities, at HBOI, FL. Shrimp (0.8-1.2 g body weight) were transferred into 10 gallon tanks containing filtered marine water (10 shrimp per tank). 5 tanks for each experimental treatment were used. Constant aeration and commercial diet were provided maintaining the following conditions: salinity 30 ppt, pH 8.0; temperature, 28±1.0° C. The experimental design comprised the following experimental groups: (i) negative control, uninfected (only seawater), (ii) Bacillus BCG322 (pAD PB-Sr) administration before V. parahaemolyticus infection challenge, and (iii) positive control infected Bacillus BCG322 (pAD-luc) administration before V. parahaemolyticus infection challenge. The treatments consisted administering V. parahaemolyticus soaked pellets (1 mL/g) at a dose of 10⁹ CFU/mL.

PirA^{Vp}/PirB^{Vp} Cytotoxicity. Toxicity was determined using LDH Cytotoxicity Detection Kit. Overnight cultures of Bacillus (BCG322 (pAD PB-Sr) and BCG322 (pAD-luc)) was centrifuged at 4600 rpm per 10 min. Pellet was re-suspended pellet in 14 mL of LBS to get OD˜1. 0.5 ml of overnight V. parahaemolyticus was mixed with 10 ml of washed Bacillus culture. Bacteria mixes were incubated at 30° C. for 3, 5, 8, 17, and 24 hours with aeration. Cultural media was collected ant tested for cytotoxicity by determination of lactate dehydrogenase activity. Lactate dehydrogenase (LDH) is a stable cytoplasmic enzyme is released from cells and into the surrounding cell-culture supernatant during damage to cell cytoplasmic membranes by membrane pore-forming toxins. LDH activity in the surrounding cell-culture medium was measured by coupled reaction that converts yellow tetrazolium salt into a red formazan product. The amount of LDH enzyme activity was measured as a 490/492 nm absorbance reading on a microplate reader; it correlates with the number of damaged cells in culture.

### TABLES

TABLE 1
Strains, plasmids, and oligonucleotides.

| Strains | Genotype | Origin |
|---|---|---|
| Vibrio | pirA positive WT isolate | HBOI, Florida |
| parahaemolyticus | from infected shrimp pond | |
| Bacillus subtilis: | | |
| BCG322 | Plasmid pAD-PB-Sr | Plasmid made by |
| BCG322-PB-Sr | transformed into BCG322 | GENESCRIPT |
| | | transformed into |
| | | BCG322 |
| BCG322-pLuc | Control plasmid pAD-Luc | Lab stock |
| | transformed into BCG322 | |
| Plasmid | Description | Origin |
| pAD43-25 | Gram-positive-E. coli | [11] |
| | shuttle vector expressing | |
| | gfp underP upp, AP^{R} | |
| pAD-PB-Sr | Δ1-262 PirB^{Vp} under Pupp | Plasmid made by |
| | cloned into pAD43-25, Cm^{R} | GENESCRIPT |
| | | This invention |
| pAD-Luc | luciferase under Pupp | Inventor lab stock |
| | cloned into pAD43-25, Cm^{R} | |
| Oligonucleotides | Sequence | Purpose |
| Ap4-F1 | GTGGTAATAGATTGTACAGAA | pirA PCR detection, |
| | (SEQ ID NO. 21) | qRT-PCR and |
| | | sequencing |
| Ap3R | GTGGTAATAGATTGTACAGAA | pirA PCR detection, |
| | (SEQ ID NO. 22) | qRT-PCR and |
| | | sequencing |
| Vp-gyrB-for | CGAGCATGCGCTAAGTGTTG | qPCR of gyrB |
| | (SEQ ID NO. 23) | house-keeping gene |
| Vp-gyrB-rev | TAACGCTGACGGCTTAGACC | qPCR of gyrB |
| | (SEQ ID NO. 24) | house-keeping gene |

TABLE 2
Computationally selected PB-Sr (PirB^{Vp} -Δ1-262)
variants with enhanced affinity to PirA^{Vp}

| | Predicted binding affinity |
|---|---|
| PirB^{Vp}-Δ1-262 construct | (K_{d}; nM units) |
| WT | 7330 (experimental) |
| F276S | 1354 |
| A367T | 1604 |
| P395Y | 1899 |
| F276S/A367T | 296 |
| F276S/P395Y | 351 |
| A367T/P395Y | 415 |
| F276S/A367T/P395Y | 76 |

TABLE 3
Computationally selected PirB^{Vp}-derived
peptides tob e used as competitors of
PirB^{Vp}/PirA^{Vp} interactions.

| | Predicted |
|---|---|
| | binding affinity |
| PirB^{Vp}-based peptides | (K_{d}; nM units) |
| 214-WADNDSYNNANQDNVYDEVMGAR-236 | 19 |
| (SEQ ID NO. 16) | |
| 214-WADNDSYNNANQD-226 | 577 |
| (SEQ ID NO. 17) | |
| 386-FVVGENSGKPSVRLQL-401 | 895 |
| (SEQ ID NO. 18) | |
| 392-SGKPSVRLQL-401 | 1416 |
| (SEQ ID NO. 19) | |

### REFERENCES

1. Kumar, R., T. H. Ng, and H.-C. Wang, Acute hepatopancreatic necrosis disease in penaeid shrimp. Reviews in Aquaculture, 2020. 12(3): p. 1867-1880.
2. Tran, L., et al., Determination of the infectious nature of the agent of acute hepatopancreatic necrosis syndrome affecting penaeid shrimp. Dis Aquat Organ, 2013. 105(1): p. 45-55.
3. Kushwah, J. and V. S. Somvanshi, Photorhabdus: A Microbial Factory of Insect-Killing Toxins, in Microbial Factories: Biodiversity, Biopolymers, Bioactive Molecules: Volume 2, V. C. Kalia, Editor. 2015, Springer India: New Delhi. p. 235-240.
4. Sirikharin, R., et al., Characterization and PCR Detection Of Binary, Pir-Like Toxins from Vibrio parahaemolyticus Isolates that Cause Acute Hepatopancreatic Necrosis Disease (AHPND) in Shrimp. PLoS One, 2015. 10(5): p. e0126987.
5. Lee, C. T., et al., The opportunistic marine pathogen Vibrio parahaemolyticus becomes virulent by acquiring a plasmid that expresses a deadly toxin. Proc Natl Acad Sci USA, 2015. 112(34): p. 10798-803.
6. Lin, S. J., et al., Structural Insights to the Heterotetrameric Interaction between the Vibrio parahaemolyticus PirA(vp) and PirB(vp) Toxins and Activation of the Cry-Like Pore-Forming Domain. Toxins (Basel), 2019. 11(4).
7. de Vries, S. J. and A. M. J. J. Bonvin, CPORT: a Consensus Interface Predictor and its Performance in Prediction-Driven Docking with HADDOCK. PLoS One, 2011. 6: p. e17695.
8. Schymkowitz, J., et al., The FoldX web server: an online force field. Nucleic Acids Res, 2005. 33: p. W382-8.
9. Xue, L., et al., PRODIGY: a web-server for predicting the binding affinity in protein-protein complexes. Bioinformatics, 2016. 32: p. 3676-8.
10. Kurcinski, M., et al., Flexible docking of peptides to proteins using CABS-dock. Protein Sci, 2020. 29: p. 211-22.
11. Dunn, A. K. & J. Handelsman, (1999) A vector for promoter trapping in Bacillus cereus. Gene 226: 297-305.
12. Lin S J, Hsu K C, Wang H C. Structural Insights into the Cytotoxic Mechanism of Vibrio parahaemolyticus PirA(vp) and PirB(vp) Toxins. Mar Drugs. 2017 Dec. 1; 15(12).

SEQUENCE LISTING

SEQ ID NO. 1
Amino Acid
PirA
Vibrio parahaemolyticus
MSNNIKHETDYSHDWTVEPNGGVTEVDSKHTPIIPEVGRSVDIENTGRGELTIQYQWGAPFMAGGWKVAK
SHVVQRDETYHLQRPDNAFYHQRIVVINNGASRGFCTIYYH
SEQ ID NO. 2
Amino Acid
PirB-WT
Vibrio parahaemolyticus
MTNEYVVTMSSLTEFNPNNARKSYLFDNYEVDPNYAFKAMVSFGLSNIPYAGGFLSTLWNIFWPNTPNEP
DIENIWEQLRDRIQDLVDESIIDAINGILDSKIKETRDKIQDINETIENFGYAAAKDDYIGLVTHYLIGL
EENFKRELDGDEWLGYAILPLLATTVSLQITYMACGLDYKDEEGETDSDVHKLTRNIDKLYDDVSSYITE
LAAWADNDSYNNANQDNVYDEVMGARSWCTVHGFEHMLIWQKIKELKKVDVFVHSNLISYSPAVGFPSGN
FNYIATGTEDEIPQPLKPNMFGERRNRIVKIESWNSIEIHYYNRVGRLKLTYENGEVVELGKAHKYDEHY
QSIELNGAYIKYVDVIANGPEAIDRIVEHFSDDRTFVVGENSGKPSVRLQLEGHFICGMLADQEGSDKVA
AFSVAYELFHPDEFGTEK
SEQ ID NO. 3
Amino Acid
PirB^{Vp} Δ1-262
Vibrio parahaemolyticus
VHSNLISYSPAVGFPSGNFNYIATGTEDEIPQPMKPNMFGERRNRIVKIESWNSIEIHYYNRVGRLKLTY
ENGEVVELGKAHKYDEHYQSIELNGAYIKYVDVIANGPEAIDRIVFHFSDDRTFVVGENSGKPSVRLQLE
GHFICGMLADQEGSDKVAAFSVAYELFHPDEFGTEK
SEQ ID NO. 4
Amino Acid
PirB^{Vp} Δ1-262-Ybxf
Vibrio parahaemolyticus
MKKWIYVVLVLSIAGIGGFSVHAVHSNLISYSPAVGFPSGNFNYIATGTEDEIPQPLKPNMFGERRNRIV
KIESWNSIEIHYYNRVGRLKLTYENGEVVELGKAHKYDEHYQSIELNGAYIKYVDVIANGPEAIDRIVFH
FSDDRTFVVGENSGKPSVRLQLEGHFICGMLADQEGSDKVAAFSVAYELFHPDEFGTEK
SEQ ID NO. 5
Amino Acid
PirB^{Vp} Δ1-262-F276S
Vibrio parahaemolyticus
VHSNLISYSPAVGSPSGNFNYIATGTEDEIPQPLKPNMFGERRNRIVKIESWNSIEIHYYNRVGRLKLTY
ENGEVVELGKAHKYDEHYQSIELNGAYIKYVDVIANGPEAIDRIVEHFSDDRTFVVGENSGKPSVRLQLE
GHFICGMLADQEGSDKVAAFSVAYELFHPDEFGTEK
SEQ ID NO. 6
Amino Acid
PirB^{Vp} Δ1-262-A367T
Vibrio parahaemolyticus
VHSNLISYSPAVGFPSGNFNYIATGTEDEIPQPLKPNMFGERRNRIVKIESWNSIEIHYYNRVGRLKLTY
ENGEVVELGKAHKYDEHYQSIELNGAYIKYVDVITNGPEAIDRIVFHFSDDRTFVVGENSGKPSVRLQLE
GHFICGMLADQEGSDKVAAFSVAYELFHPDEFGTEK
SEQ ID NO. 7
Amino Acid
PirB^{Vp} Δ1-262-P395Y
Vibrio parahaemolyticus
VHSNLISYSPAVGFPSGNFNYIATGTEDEIPQPLKPNMFGERRNRIVKIESWNSIEIHYYNRVGRLKLTY
ENGEVVELGKAHKYDEHYQSIELNGAYIKYVDVIANGPEAIDRIVFHFSDDRTFVVGENSGKYSVRLQLE
GHFICGMLADQEGSDKVAAFSVAYELFHPDEFGTEK
SEQ ID NO. 8
Amino Acid
PirB^{Vp} Δ1-262-F276S/A367T
Vibrio parahaemolyticus
VHSNLISYSPAVGSPSGNFNYIATGTEDEIPQPLKPNMFGERRNRIVKIESWNSIEIHYYNRVGRLKLTY
ENGEVVELGKAHKYDEHYQSIELNGAYIKYVDVITNGPEAIDRIVFHFSDDRTFVVGENSGKPSVRLQLE
GHFICGMLADQEGSDKVAAFSVAYELFHPDEFGTEK
SEQ ID NO. 9
Amino Acid
PirB^{Vp} Δ1-262-F276S/P395Y
Vibrio parahaemolyticus
VHSNLISYSPAVGSPSGNFNYIATGTEDEIPQPLKPNMFGERRNRIVKIESWNSIEIHYYNRVGRLKLTY
ENGEVVELGKAHKYDEHYQSIELNGAYIKYVDVIANGPEAIDRIVFHFSDDRTFVVGENSGKYSVRLQLE
GHFICGMLADQEGSDKVAAFSVAYELFHPDEFGTEK
SEQ ID NO. 10
Amino Acid
PirB^{Vp} Δ1-262-A367T/P395Y
Vibrio parahaemolyticus
VHSNLISYSPAVGFPSGNFNYIATGTEDEIPQPLKPNMFGERRNRIVKIESWNSIEIHYYNRVGRLKLTY
ENGEVVELGKAHKYDEHYQSIELNGAYIKYVDVITNGPEAIDRIVFHFSDDRTFVVGENSGKYSVRLQLE
GHFICGMLADQEGSDKVAAFSVAYELFHPDEFGTEK
SEQ ID NO. 11
Amino Acid
PirB^{Vp} Δ1-262-F276S/A367T/P395Y
Vibrio parahaemolyticus
VHSNLISYSPAVGSPSGNFNYIATGTEDEIPQPLKPNMFGERRNRIVKIESWNSIEIHYYNRVGRLKLTY
ENGEVVELGKAHKYDEHYQSIELNGAYIKYVDVITNGPEAIDRIVFHFSDDRTFVVGENSGKYSVRLQLE
GHFICGMLADQEGSDKVAAFSVAYELFHPDEFGTEK
SEQ ID NO. 12
DNA
PirB^{Vp} Δ1-262
Vibrio parahaemolyticus
GTTCACAGTAATTTAATTTCATATTCACCTGCTGTTGGTTTTCCTAGTGGTAATTTCAACTATATTGCTA
CAGGTACGGAAGATGAAATACCTCAACCATTAAAACCAAATATGTTTGGGGAACGTCGAAATCGTATTGT
AAAAATTGAATCATGGAACAGTATTGAAATACATTATTACAATCGCGTAGGTCGACTTAAACTAACTTAT
GAAAATGGGGAAGTGGTAGAACTAGGCAAGGCTCATAAATATGACGAGCATTACCAATCTATTGAGTTAA
ACGGCGCTTACATTAAATATGTTGATGTTATTGCCAATGGACCTGAAGCAATTGATCGAATCGTATTTCA
TTTTTCAGATGATCGAACATTTGTTGTTGGTGAAAACTCAGGCAAGCCAAGTGTGCGTTTGCAACTGGAA
GGTCATTTTATTTGTGGCATGCTTGCGGATCAAGAAGGTTCTGACAAAGTTGCCGCGTTTAGCGTGGCTT
ATGAATTGTTTCATCCCGATGAATTTGGTACAGAAAAGTAG
SEQ ID NO. 13
DNA
PirB vP PB-Sr-YbxI
Vibrio parahaemolyticus
ATGAAAAAATGGATATATGTTGTGCTTGTGCTGAGTATTGCAGGGATCGGCGGCTTCTCCGTCCACGCAG
TTCACAGTAATTTAATTTCATATTCACCTGCTGTTGGTTTTCCTAGTGGTAATTTCAACTATATTGCTAC
AGGTACGGAAGATGAAATACCTCAACCATTAAAACCAAATATGTTTGGGGAACGTCGAAATCGTATTGTA
AAAATTGAATCATGGAACAGTATTGAAATACATTATTACAATCGCGTAGGTCGACTTAAACTAACTTATG
AAAATGGGGAAGTGGTAGAACTAGGCAAGGCTCATAAATATGACGAGCATTACCAATCTATTGAGTTAAA
CGGCGCTTACATTAAATATGTTGATGTTATTGCCAATGGACCTGAAGCAATTGATCGAATCGTATTTCAT
TTTTCAGATGATCGAACATTTGTTGTTGGTGAAAACTCAGGCAAGCCAAGTGTGCGTTTGCAACTGGAAG
GTCATTTTATTTGTGGCATGCTTGCGGATCAAGAAGGTTCTGACAAAGTTGCCGCGTTTAGCGTGGCTTA
TGAATTGTTTCATCCCGATGAATTTGGTACAGAAAAGTAG
SEQ ID NO. 14
Amino Acid
YbxI
Bacillus subtilis
MKKWIYVVLVLSIAGIGGFSVHA
SEQ ID NO. 15
DNA
YbxI
Bacillus subtilis
ATGAAAAAATGGATATATGTTGTGCTTGTGCTGAGTATTGCAGGGATCGGCGGCTTCTCCGTCCACGCA
SEQ ID NO. 16
Amino Acid
PirB Peptide 214-236
Vibrio parahaemolyticus
WADNDSYNNANQDNVYDEVMGAR
SEQ ID NO. 17
Amino Acid
PirB Peptide 214-226
Vibrio parahaemolyticus
WADNDSYNNANQD
SEQ ID NO. 18
Amino Acid
PirB Peptide 386-401
Vibrio parahaemolyticus
FVVGENSGKPSVRLQL
SEQ ID NO. 19
Amino Acid
PirB Peptide 392-401
Vibrio parahaemolyticus
SGKPSVRLQL
SEQ ID NO. 20
Amino Acid
Epitope fragment of pirA
Vibrio parahaemolyticus
CVQRDETYHLQRPDN
SEQ ID NO. 21
DNA
Ap4-F1
Artificial
GTGGTAATAGATTGTACAGAA
SEQ ID NO. 22
DNA
Ap3R
Artificial
GTGGTAATAGATTGTACAGAA
SEQ ID NO. 23
DNA
Vp-gyrB for primer
Artificial
CGAGCATGCGCTAAGTGTTG
SEQ ID NO. 24
DNA
Vp-gyrB-rev
Artificial
TAACGCTGACGGCTTAGACC

1. A composition for the treatment of Early Mortality Syndrome (EMS) in shrimp comprising a modified PirB peptide, wherein said modified PirB peptide competitively inhibits the formation of the PirA/PirB dimer complex.

2. The composition of claim 1, wherein said modified PirB peptide comprises a truncated PirB peptide.

3. The composition of claim 2, wherein said truncated PirB peptide comprises a PirB Δ1-262 peptide.

4. The composition of claim 2, wherein said truncated PirB peptide comprises the amino acid sequence according to SEQ ID NO. 3.

5. The composition of claim 2, wherein said truncated PirB peptide is coupled with a secretion signal domain.

6. (canceled)

7. The composition of claim 2, wherein said truncated PirB peptide coupled with a secretion signal domain comprises the amino acid sequence according to SEQ ID NO. 4.

8. The composition of claim 5, wherein said secretion signal domain comprises a secretion signal according to SEQ ID NO. 14.

9. The composition of claim 2, wherein the truncated PirB peptide further comprises a truncated PirB peptide having one or more mutations selected from the group consisting of: F276S, A367T, P395Y, or any combination thereof.

10. The composition of claim 2, wherein the truncated PirB peptide further comprises a truncated PirB peptide selected from the group consisting of: SEQ ID NOs. 5-11.

11. The composition of claim 1, wherein said PirA/PirB complex comprises a dimer complex wherein PirA comprises a sequence according to SEQ ID NO. 1, and PirB comprises a sequence according to SEQ ID NO. 2.

12-34. (canceled)

35. A composition for the treatment of Early Mortality Syndrome (EMS) in an aquatic organism comprising a PirB peptide fragment, wherein said PirB peptide fragment competitively inhibits the formation of the PirA/PirB dimer complex.

36. The composition of claim 35, wherein said PirB peptide fragment comprises a PirB peptide fragment encoding a portion of a binding interface with PirA.

37. The composition of claim 36, wherein said PirB peptide fragment comprises a PirB peptide fragment selected from the group consisting of: SEQ ID NOs. 16-19.

38. The composition of claim 36, wherein said PirB peptide fragment is coupled with a secretion signal domain through a linker domain.

39. The composition of claim 38, wherein said secretion signal domain comprises a secretion signal according to SEQ ID NO. 14.

40. The composition of claim 35, wherein said PirA/PirB complex comprises a dimer complex wherein PirA comprises a sequence according to SEQ ID NO. 1, and PirB comprises a sequence according to SEQ ID NO. 2.

41-69. (canceled)

70. A composition for the treatment of Early Mortality Syndrome (EMS) comprising a PirB peptide fragment, wherein said PirB peptide fragment competitively inhibits the formation of the PirA/PirB dimer complex; wherein said PirB peptide further comprises a truncated PirB peptide selected from the group consisting of: SEQ ID NO's. 3, and 5-11, or a combination of the same.

71. The composition of claim 2, wherein said truncated PirB is expressed in a bacteria administered to a shrimp.

72. The composition of claim 36, wherein said PirB peptide fragment is expressed in a bacteria administered to a shrimp.

73. The composition of claim 70, wherein said truncated PirB is expressed in a bacteria administered to a shrimp.
